(19) United States
(12) Patent Application Publication
(10) Pub. No.: US 2004/0072882 A1
(43) Pub. Date: Apr. 15, 2004

(54) Methods to administer epothilone D

(75) Inventors: Robert G. Johnson JR., Lafayette, CA (US); Michael J. Sherrill, Danville, CA (US); Alison Hannah, Sebastopol, CA (US)

Correspondence Address: David P. Lentini, Kosan Biosciences, Inc., 3832 Bay Center Place, Hayward, CA, 94545, US

(73) Assignee: Kosan Biosciences, Inc., A Delaware Corporation, Hayward, CA (US)

(21) Appl. No.: 10/442,820

(22) Filed: May 20, 2003

Related U.S. Application Data
(60) Provisional application No. 60/382,166, filed on May 20, 2002.

(51) Int. Cl. A61K 31/427

(52) U.S. Cl. 514/365

(57) ABSTRACT

Methods to deliver epothilone D to subjects having tumorigenic diseases are provided. In some embodiments, the invention provides methods for treating tumor-bearing subjects with an intravenous infusion of epothilone D at least once about every seven days throughout a delivery period of about twenty-one consecutive day period

### 1 CROSS REFERENCE TO RELATED U.S. PATENT APPLICATIONS

This patent application claims priority under 35 U.S.C. § 119(e) as a continuation-in-part of Provisional U.S. Patent Application Serial No. 60/382,166, which is incorporated herein by reference for all purposes.

### 2 BACKGROUND OF THE INVENTION

2.1 Field of the Invention

The instant invention relates to the treatment of proliferative diseases, and, especially, cancer. More specifically, the present invention provides methods to administer epothilones, and, more specifically, epothilone D, to achieve a therapeutic effect. The instant invention thus has relevance to the fields of medicine, oncology, and pharmacology.

2.2 The Related Art

The class of ketolides known as epothilones has emerged as a source of potentially therapeutic compounds having modes of action similar to paclitaxel (Bollag, et al. 1995; Service 1996; Winkler and Axelsen 1996; Bollag 1997; Cowden and Paterson 1997). Interest in the epothilones and epothilone analogs has grown with the observations that certain epothilones are active against tumors that have developed resistance to paclitaxel (Harris, et al. 1999a) as well as reduced potential for undesirable side-effects (Muhlradt and Sasse 1997). Among the epothilones and epothilone analogs being investigated for therapeutic efficacy are epothilone B 1 (Oza, et al. 2000) and the semi-synthetic epothilone B analogs, BMS-247550 2, also known as “azaepothilone B” (Colevas, et al. 2001; Lee, et al. 2001; McDaid, et al. 2002; Yamaguchi, et al. 2002), and BMS-310705 3

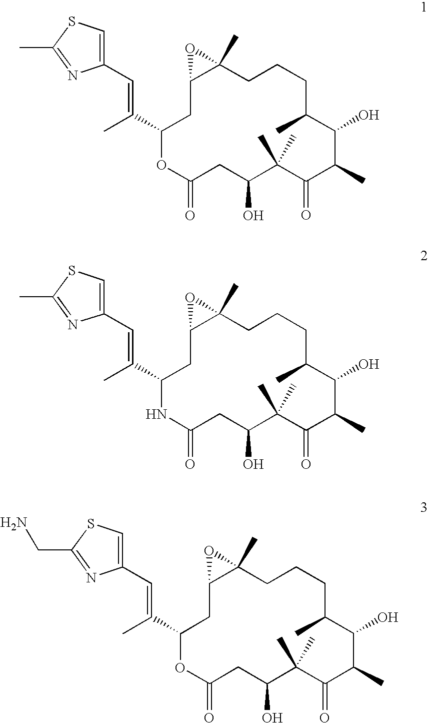

Desoxyepothilone B 4, also known as “epothilone D”, is another epothilone derivative having promising antitumor properties vis àvis. paclitaxel that is being investigated for therapeutic efficacy (Su, et al. 1997; Chou, et al. 1998a; Chou, et al. 1998b; Harris, et al. 1998b; Chou, et al. 2001; Danishefsky, et al. 2001; Martin and Thomas 2001; Danishefsky, et al. 2002). This compound has also demonstrated less toxicity than epothilones having 12,13-epoxides, such as epothilone B or BMS-247550, presumably due to the lack of the highly reactive epoxide moiety.

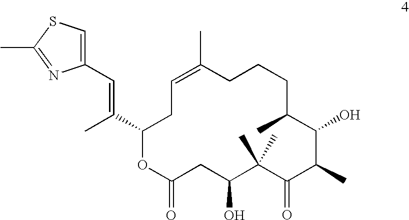

Clinicians seek dosages and administration schedules for delivering drugs to a patient that are both effective and tolerable. Often those having skills in the clinical arts must find a dose and schedule that balances the toxicity of a drug with the drug's therapeutic effect. U.S. Pat. Nos. 6,641,803 and 5,635,531 describe such dosing regimens for paclitaxel; and U.S. Pat. No. 6,302,838 illustrates dosing regimens for epothilone B. However, an optimal dosing regimen for epothilone D remains to be determined.

### 3 SUMMARY OF THE INVENTION

In one aspect, the present invention provides methods for delivering epothilone D to a tumor-bearing subject. According to one embodiment of the invention, the subject receives a therapeutically effective amount of epothilone D by intravenous infusion. In some embodiments, the epothilone D is delivered in a concentration of between about 0.25 mg/mL and about 2.0 mg/mL. In other such embodiments, the epothilone D is delivered in a concentration of between about 0.5 mg/mL and about 1.0 mg/mL. The dose of epothilone D can be at least about 100 mg of epothilone D per square meter of the subject's surface area.

In another aspect, the intravenous infusion is performed in a treatment cycle that includes infusing the subject at least once about every seven days throughout a delivery period of about twenty-one consecutive days. In other embodiments, the infusion is performed twice over about fourteen days during the delivery period. In more specific embodiments of either case, the treatment cycle has a duration of about twenty-eight days. Still other embodiments of the method of the invention include those for which the treatment cycle is repeated.

In still another aspect, the intravenous infusion is performed in a treatment cycle in which the infusion is performed once about every twenty-four hours throughout a delivery period of about seventy-two hours. In some more specific embodiments, the treatment cycle has a duration of about seven consecutive days. In still more specific embodiments, at least about 40 mg of epothilone D per square meter is delivered. In yet more specific embodiments, the infusion is performed over a period of less than about two hours.

In another aspect, the intravenous infusion is performed continuously for a period of about twenty-four hours. In some embodiments of this aspect of the invention, a loading dose is provided to the subject. In more specific embodiments, the loading dose is followed by a continuous infusion.

These and other aspects and advantages will become apparent when the Description below is read in conjunction with the accompanying Drawings.

### 4 BRIEF DESCRIPTION OF THE DRAWINGS

FIG. 1A and FIG. 1B show the concentration of epothilone D in the plasma of subjects as a function of time. FIG. 1A shows the results in nanograms per milliliter (ng/mL) of plasma as a function of time. FIG. 1B shows a comparison of the results obtained in two different cycles for three subjects.

FIG. 2 is a graph of the area under the curve (AUC), the total exposure of epothilone D experienced by the patient as a function of dose.

FIG. 3 is graph showing the formation of microtubule bundles bound by epothilone D as a function of time.

FIG. 4A and FIG. 4B show the relationship of pharmacodynamics and end-infusion concentration of epothilone D. FIG. 4A shows the relationship for bundle formation. FIG. 4B shows the relationship for AUC.

FIG. 5 shows efficacy for a patient treated according to the method of the invention.

### DESCRIPTION OF SOME EMBODIMENTS OF THE INVENTION

The present invention provides methods to administer epothilone D as an antitumor treatment. In one aspect, the invention provides a method to provide an antitumor treatment to a tumor-bearing subject, comprising: administering a composition containing a therapeutically effective amount of epothilone D to such subject by intravenous infusion. The epothilone D delivered using the methods of the invention can be formulated using physiological saline or alternative aqueous media for administration to subjects using agents to enhance the solubility of the epothilone D, as will be familiar to one of skill in the pharmaceutical arts (Gennaro 2000). One example of such an agent is CREMOPHOR®. For example, as detailed in the exemplified protocols below, one suitable preparation administered successfully to subjects contains 1% CREMOPHOR® and 0.5 mg epothilone D per milliliter (mL) of solution. Higher or lower amounts of epothilone D for example, in the range of about 0.25 mg epothilone D to about 2.0 mg of epothilone D per mL, can also be used. As will be familiar to those of skill in the pharmaceutical arts, some agents that are effective to enhance the solubility of epothilone D, such as CREMOPHOR®, may induce negative reactions when given to subjects, and, therefore, drugs to counteract such negative reactions may be administered along with, after, or prior to, administration of the epothilone D as described herein. Alternative, CREMOPHOR®-free, formulations are described in co-pending Provisional U.S. Patent Applications Serial Nos. 60/417,356 and 60/426,585; each of these pending applications is incorporated herein by reference for all purposes.

The above-described formulations can be delivered using methods and materials known to those having skill in the pharmaceutical and medical arts with appropriate adjustment of infusion rate and time of infusion. Generally, intravenous administration by infusion using a dosage rate that is approximately 150 cubic centimeters (cc) of infusate per hour (150 cc/hr). In other embodiments, the infusion is performed over ninety minutes, and, in still other embodiments, the formulation is delivered by a first, relatively rapid (e.g., over a period of about thirty minutes) loading dose followed by steady, low-dose infusion (e.g., delivered over a period of between twenty-four to seventy-two hours). The time for infusion will in general depend on the dosage. A general range of infusion times is between about ten minutes to about ten hours; but in most cases infusion time will not exceed about six hours, and, in some cases, the infusion time will not exceed two hours. Alternatively, a preset time for infusion of between about thirty and about ninety minutes is fixed, and the rate of infusion is adjusted accordingly thereto.

In order to ensure that toxic limits are not exceeded, the effects of the administration on the subject are monitored. Possible effects include neurological impairment(s), which may manifest itself as cognitive/perceptual abnormalities, numbness in the limbs, difficulty in walking, dizziness, and the like. For example, at a dose level of between nine milligrams (mg) of epothilone D and about sixty milligrams per unit area of the subjects surface (in square meters (m²)) toxicity will typically start at day five and continue to day 15; however, at higher dosages such as 90 mg/m² and 185 g/m², toxicity can begin as soon as the day after infusion is terminated. Other side effects may include nausea and vomiting, fatigue, rash, alopecia, and alteration in vital signs such as orthostatic hypotension. Myelosuppression (which may manifest itself as anemia, neutropenia, thrombocytopenia, and the like) should also be monitored, although myelosuppression has generally not been seen with this drug.

In some embodiments, the present invention provides a method to provide an antitumor treatment to a tumor-bearing subject. In one embodiment, the method of invention includes administering a composition containing a therapeutically effective amount of epothilone D to such subject by intravenous infusion. In a more specific embodiment, the concentration of epothilone D in the composition delivered by intravenous infusion is between about 0.25 mg/mL and about 2.0 mg/mL; in another embodiment, the concentration of epothilone D in the composition is between about 0.5 mg/mL and about 1.0 mg/mL; and, in a still more specific embodiment, the concentration of epothilone D in the composition is about 0.5 mg/mL. The dose of epothilone D delivered to the subject by intravenous infusion is generally less than about 250 milligrams per square meter of the subject's surface area (250 mg/m²), and, more specifically, between about 70 mg/m² and about 250 mg/m². In some embodiments, the dose delivered is at least about 100 mg of epothilone D per square meter of the surface area of such subject, and, in more particular embodiments, at least about 120 mg of epothilone D per square meter of the surface area of such subject. Yet more specific dosing ranges of epothilone D according to some embodiments of the invention are between about 100 mg/m² and about 200 mg/m². In other embodiments, the period for dosing by intravenous infusion is less than about 6 hours.

In another embodiment, the invention provides a treatment cycle comprising performing the step of administering by intravenous infusion at least once about every seven days throughout a delivery period of about twenty-one consecutive days. In a more specific embodiment, the treatment cycle just described further includes repeating the step of administering by intravenous infusion twice over about fourteen days throughout the delivery period of about twenty-one consecutive days. Still another embodiment of the cycle, including either the single intravenous infusion once about seven days or the embodiment in which separate infusions at once per seven days are given twice in a twenty-one day period, further include the step of evaluating the status of such subject to determine whether to administer additional epothilone D to such subject. In another embodiment of these embodiments just described, the treatment cycle has a duration of about twenty-eight days. In more specific embodiments including the twenty-eight-day treatment cycle, the delivery period begins on the first day of said treatment cycle; and, in a still more specific embodiment of the the twenty-eight-day treatment cycle in which delivery period begins on the first day of said treatment cycle, the invention further includes the step of repeating the treatment cycle after the completion of the treatment period.

Further, more specific embodiments, of those embodiments including twenty-one day intravenous delivery periods include those for which the concentration of epothilone D in the composition delivered by intravenous infusion is between about 0.25 mg/mL and about 2.0 mg/mL; in another embodiment, the concentration of epothilone D in the composition is between about 0.5 mg/mL and about 1.0 mg/mL; and, in a still more specific embodiment, the concentration of epothilone D in the composition is about 0.5 mg/mL. The dose of epothilone D delivered to the subject by intravenous infusion is generally less than about 250 milligrams per square meter of the subject's surface area (250 mg/m²), and, more specifically, between about 70 mg/m² and about 250 mg/m². In some embodiments, the dose delivered is at least about 100 mg of epothilone D per square meter of the surface area of such subject, and, in more particular embodiments, at least about 120 mg of epothilone D per square meter of the surface area of such subject. Yet more specific dosing ranges of epothilone D according to some embodiments of the invention are between about 100 mg/m² and about 200 mg/m². In other embodiments, the period for dosing by intravenous infusion is less than about 6 hours.

In still other embodiments, the method of invention includes administering a composition containing a therapeutically effective amount of epothilone D to such subject by intravenous infusion in a treatment cycle comprising performing the step of administering by intravenous infusion once about every twenty-four hours throughout a delivery period of about seventy-two hours. In more specific embodiments in which a seventy-two-hour delivery period is used, the treatment cycle has a duration of about fourteen consecutive days. Still more specific embodiments in which a seventy-two hour delivery period is used and the treatment cycle has a duration of about fourteen consecutive days include those for which the treatment cycle is repeated two times over about twenty-eight consecutive days. According to some embodiments in which the treatment cycle is repeated two times over about twenty-eight consecutive days, the intravenous infusion is performed over a period of less than about two hours. Still more specific embodiments of either of the latter two embodiments include those for which the amount of epothilone D administered to the subject is at least about 40 mg of epothilone D per square meter of the surface area of such subject; in yet more specific embodiments the amount of epothilone D administered to the subject is at least about 50 mg of epothilone D per square meter of the surface area of such subject.

Of the several embodiments just described for which the method of invention includes administering a composition containing a therapeutically effective amount of epothilone D to such subject by intravenous infusion in a treatment cycle comprising performing said step of administering by intravenous infusion once about every twenty-four hours throughout a delivery period of about seventy-two hours, more specific embodiment include those for which the concentration of epothilone D in the composition delivered by intravenous infusion is between about 0.25 mg/mL and about 2.0 mg/mL, the concentration of epothilone D in the composition is between about 0.5 mg/mL and about 1.0 mg/mL; and, still more specifically, the concentration of epothilone D in the composition is about 0.5 mg/mL.

Yet other embodiments described for which the method of invention includes administering a composition containing a therapeutically effective amount of epothilone D to such subject by intravenous infusion include those for which the infusion is performed continuously for a period of about twenty-four hours. Such embodiments, further includes those including providing a loading dose, and, more specific embodiments in which the just-described loading dose is performed for about thirty minutes. In addition, the dose of epothilone D delivered using any of these embodiments that include twenty-four-hour continuous dosing can be less than about 250 mg and, more specifically about 70 mg or about 200 mg

In general, the pharmacokinetics of epothilone D administration are favorable. As described below, the exposure determined for epothilone D administration were dose-dependent; and the dependence of the area under the curve (AUC) on dosage was linear for a dose range of between about 9 mg/m² and about 150 mg/m². The half-life of epothilone D had a mean value of approximately 8-10 hours, and a volume of distribution (Vz) of between 90 L/m² and 150 L/m², indicating good drug penetration. This is somewhat higher on average than the values for paclitaxel, which are 140±70 L/m². These pharmacokinetic parameters do not change appreciably for a second infusion as compared to a first infusion.

The activity of the drug can be assessed by measuring bundling of microtubules in interphase cells. This is considered the hallmark of activity of microtubule-stabilizing agents such as paclitaxel. The bundle formation can readily be measured by immunofluorescence or Western blotting. In a typical determination, whole blood is collected from patients and mononuclear cells (PBMC's) are isolated for evaluation of bundle formation. Substantial amounts of bundle formation have been observed when the dosage was as low as 18 mg/m ² and this increases with dosage. Maximum microtubule bundle formation was observed at doses of 60 mg/m²-185 mg/m².

In addition to the foregoing, the methods described herein can be used to deliver epothilone D when used in combination with other treatment modalities, including drugs, surgery, and radiation. In a more particular embodiment, the methods of the invention can be used to deliver epothilone D in combination with a nucleoside analog as described in co-pending Provisional U.S. Patent Application Serial No. 60/417,535, which is incorporated herein by reference for all purposes. In some of these embodiments, the nucleoside analog is selected from the group consisting of: azacitidine, cladribine, cytarabine, floxuridine, fludarabine phosphate, 5-fluorouracil, gemcitabine, pentostatin, uracil mustard, and 5′-deoxy-5-fluoro-N-[(pentyloxy)carbonyl]-cytidine (sold under the trade name ZELODA® (Roche).

### 6 EXAMPLES

The following Examples are provided to illustrate certain aspects of the present invention and to aid those of skill in the art in the art in practicing the invention. These Examples are in no way to be considered to limit the scope of the invention in any manner.

### 6.1 Example 1

### Patient Study

6.1.1 Enrollment

Patients were enrolled if they exhibited advanced malignancy, either primary or metastatic, were refractory to standard therapy (or no standard therapy is available) and if the last dose of anticancer therapy they had experienced, if relevant, was more than 21 days prior to enrollment. To be included in the study, the patient must have had a type of cancer that was measurable or capable of evaluation. Other criteria included adequate liver, renal and hematopoietic function, i.e., recovery from reversible effects of previous therapies, if any. Subjects included in the study provided their informed consent. Candidate patients were rejected, however, if they were allergic to CREMOPHOR®-containing products since the composition used in this study contained 0.5%-1% CREMOPHOR® as a solubilizing agent for epothilone D. Patients were also rejected if they had any preexisting neuropathy, or showed RT more than 25% bone marrow-containing skeleton, had intracranial edema or metastasis, or had epidural disease, cardiac disease, or was HIV-positive and on highly active antiretroviral therapy (HAART) regimens.

Approximately 52 patients meeting the above-described criteria were enrolled in the study. Of the patients treated, approximately 60% were male and 40% female, ranging in age from 23 to 85 years. A wide range of tumor types were included in the study, including colon, ovarian, prostate, and lung tumors. Most of the enrollees had received multiple rounds of other chemotherapies prior to entering the study.

6.1.2 Patient Dosing

H1/H2 blockers were given orally to the subjects 30-60 minutes prior to infusion to prevent any adverse reactions to the CREMOPHOR® in the composition. For each cycle, the drug was infused at a rate of about 150 cc/hr and an epothilone D concentration of about 0.5 mg/mL. Thus, a dosage of 9 mg/m² required about 10-15 minutes of infusing, while a dose of 150 mg/m² required 3-4 hours of dosing. The patients were monitored by testing CBC with differential weekly, various laboratory tests every three weeks, and physical exams including neurological assessment every three weeks. Tumor assessments were made every six weeks.

6.1.3 Results

The toxicity of epothilone D for each patient was monitored and evaluated carefully for each patient on an on-going basis during treatment. The dose-limiting toxicity was primarily neurological and was manifest by cognitive/perceptual abnormalities, which were observed only at the highest doses (i.e., between about 120 mg/m²-185 mg/m²), and which were transient. Other neurological effects included transient motor neuropathy (unsteadiness, ataxia, and dizziness), muscle twitching, and sensory neuropathy occurring as tingling with occasional numbness generally in the fingers and toes. Still other toxicities included fatigue, nausea and vomiting, diarrhea, and constipation. These toxicities were dose-dependent and generally of Grade-2 in severity. No clear evidence of myelosuppression was observed.

Both the pharmacokinetics and pharmacodynamics of epothilone D were measured in the subjects. Plasma concentration as a function of time was measured in the first- and second cycles at various dose levels in several subjects. For measuring these pharmacokinetic data, levels of epothilone D were measured prior to infusion, at 30- and 60 minutes intra-infusion if the infusion extended over this period, at the end of the infusion; and at 15-, 30-, 45-, 60-minutes and 2-, 3-, 4-, 6-, 8-, 24-, and 48 hours after infusion was terminated. Plasma analysis was performed by LC/MS/MS with a linear calibration range of 2 ng/mL-498 ng/mL; epothilone D was measured with an internal standard quantitation.

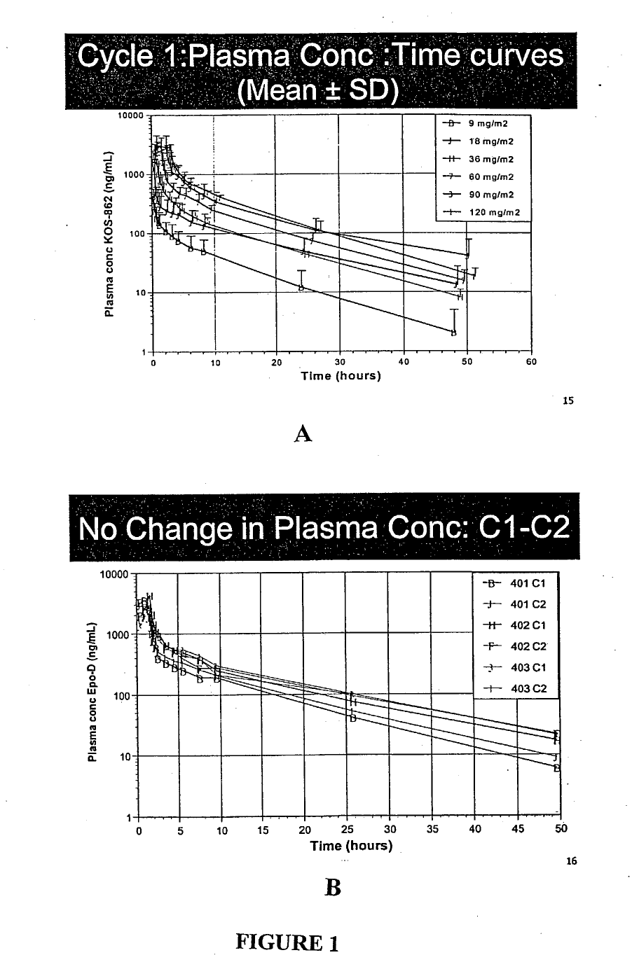

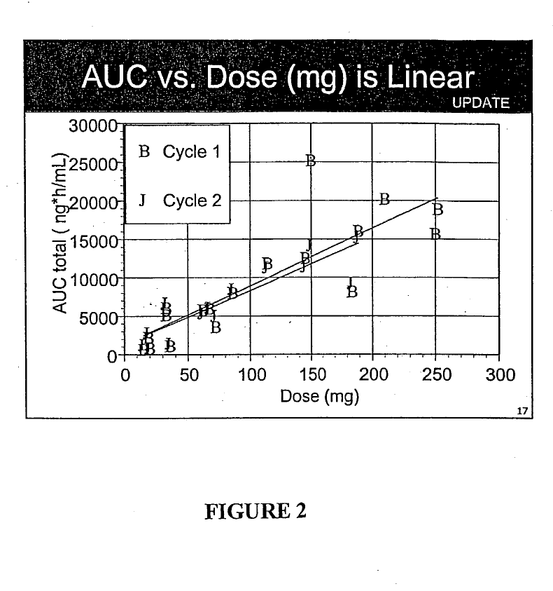

FIG. 1A shows the results in ng/ml of plasma as a function of time at a dose of 120 mg/m². As would be expected, the levels at the end of infusion are high and taper off gradually, and the concentration levels at any particular time are dose-dependent. FIG. 1B shows a comparison of the results obtained in two different cycles for three subjects treated at 60 mg/m². As shown, there is no discernable difference in pharmacokinetics based on the cycle. FIG. 2 is a graph of the area under the curve (AUC), the total amount of epothilone D experienced by the patient as a function of dose. In both first and second cycles, there is a linear correlation between the dose provided (in milligrams) and the area under the curve (which is measured in ng/ml×hours).

The results for patients treated at 100 mg/m were averaged. Drug clearance was 18.9±5.8 L/hr; the volume of distribution (Vz) was 232±82; the elimination half-life was 8.8±2.4 hr. All of these parameters were dose independent and there was no substantial change depending on the number of the cycles.

In addition to monitoring toxicity and pharmacokinetics, the pharmacodynamics of treatment was also monitored. The standard criterion is the ability of the drug to effect bundling of microtubules in interphase cells. Whole blood was collected from patients and mononuclear cells (PBMC's) were isolated. To measure bundle formation, the PBMC's were resuspended in 5% FBS/PBS containing 0.75×10⁶ cells/mL and used to make cytospin preparations. The cells were then fixed in 100% methanol for 10 minutes at 20° C., air dried and stored at 4° C. prior to immunostaining. For immunostaining, the cells were blocked in 10% Normal Goat Serum in PBS for 20 minutes and incubated with a 1:100 dilution of \tilde{□}tubulin monoclonal antibody diluted in 5% Normal Goat Serum in PBS for 1 hour at 37° C. The slides were then rinsed in PBS and incubated with 1:200 Cy3-conjugated goat anti-mouse IgG for 1 hour in the dark before mounting. Cell numbers were quantified using a Zeiss AXIOSCOP microscope and evaluated at levels of 500 cells per slide by individual investigators.

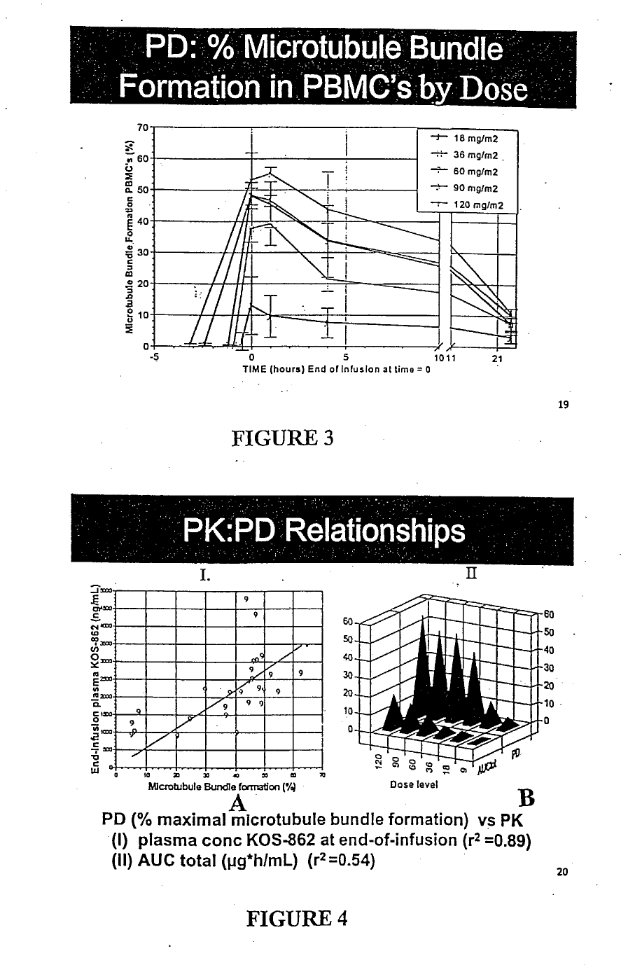

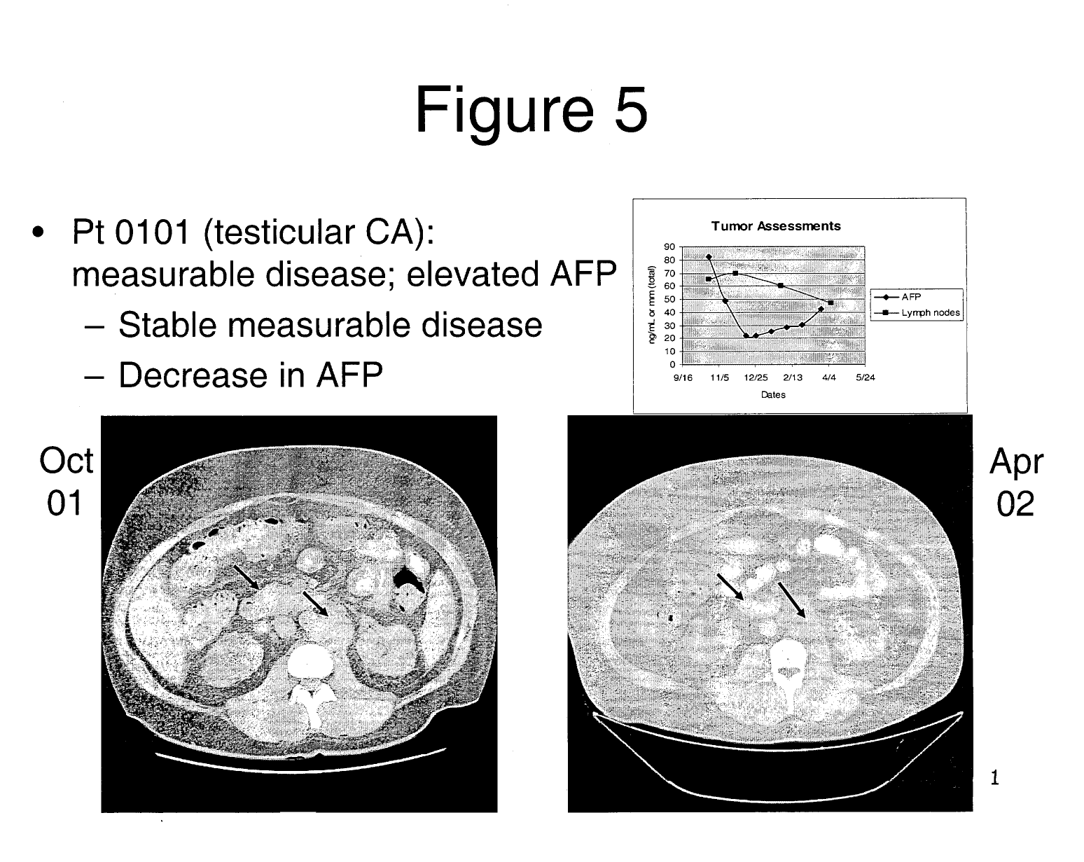

The results of evaluations of microtubule bundle formation are shown in FIG. 3. As shown, the percentage of microtubules that show bundle formation rises during the infusion and begins to taper off thereafter. The level of rise is strongly dose dependent; at a dosage of 120 mg/m², 55% of the microtubules were bundled; at 18 mg/m², only 12% of the microtubules exhibit this phenomenon.

The relationship between this pharmacodynamic effect and the concentration of epothilone D in the plasma is shown in FIG. 4. FIG. 4A shows the correlation between the concentration of epothilone D in plasma at the end of infusion and the percentage microtubule bundle formation. An excellent correlation was obtained with r²=0.89. In FIG. 4B, the correlation was made between bundle formation and area under the curve. The correlation in this case was still substantial with r²=0.54.

Tumor marker reductions were observed in several different tumor types, including: ovarian, pancreatic, testicular, breast, and biliary diseases. A number of patients received multiple cycles (at least four months), which is suggestive of stable disease.

The advantages and benefits of the invention will be apparent to those having skill in the medicine, pharmacology, and related arts. Using the methods and materials described herein, patients suffering from tumorigenic diseases can be effectively dosed to alleviate and/or eliminate their disease. Persons having skill in the arts just mentioned will also understand that the detailed description herein only illustrates the invention, but does not serve to limit the invention in any way. Indeed, persons of skill will understand that many modifications can be made to the details and examples provided herein to create other embodiments of the invention that would be within the spirit of the invention if not its literal definition. For example, dosing regimen can be provided that differ from the specifics of delivery timing, such as dosing on the sixth- or eighth day of a dosing period instead of every seventh day, without changing substantially the efficacy of the treatment methods of the invention.

### 7 BIBLIOGRAPHY

The following references are incorporated herein by reference in their entirety and for all purposes.

Bollag, D. M. (1997). “Epothilones: novel microtubule-stabilizing agents.” Expert Opinion on Investigational Drugs 6(7): 867-873.

Bollag, D. M., McQueney, P. A., et al. (1995). “Epothilones, a new class of microtubule-stabilizing agents with a taxol-like mechanism of action.” Cancer Res 55(11): 2325-33.

Chou, T. -C., O'Connor, O. A., et al. (2001). “The synthesis, discovery, and development of a highly promising class of microtubule stabilization agents: curative effects of desoxyepothilones B and F against human tumor xenografts in nude mice.” Proceedings of the National Academy of Sciences of the United States of America 98(14): 8113-8118.

Chou, T. C., Zhang, X. G., et al. (1998a). “Desoxyepothilone B is curative against human tumor xenografts that are refractory to paclitaxel.” Proc Natl Acad Sci USA 95(26): 15798-802.

Chou, T. -C., Zhang, X. -G., et al. (1998b). “Desoxyepothilone B is curative against human tumor xenografts that are refractory to paclitaxel.” Proceedings of the National Academy of Sciences of the United States of America 95(26): 15798-15802.

Colevas, A. D., West, P. J., et al. (2001). “Clinical trials referral resource. Current clinical trials of epothilone B analog (BMS-247550).” Oncology (Huntingt) 15(9): 1168-9, 1172-5.

Cowden, C. J. and Paterson, I. (1997). “Synthetic chemistry. Cancer drugs better than taxol?” Nature 387(6630): 238-9.

Danishefsky, S. J., Lee, C. B., et al. (2001). PCT Int. Appl. WO 01/64650.

Danishefsky, S. J., Stachel, S. J., et al. (2002). U.S. Pat. Appl. Publ. US 20020058286.

Gennaro, A. R., Ed. (2000). Remington: The Science and Practice of Pharmacy. Philadelphia, Lipincott Williams & Wilkins.

Harris, C. R., Balog, A., et al. (1999a). “Epothilones: microtubule stabilizing agents with enhanced activity against multidrug-resistant cell lines and tumors.” Actualites de Chimie Therapeutique 25: 187-206.

Harris, C. R., Kuduk, S. D., et al. (1999b). “New Chemical Synthesis of the Promising Cancer Chemotherapeutic Agent 12,13-Desoxyepothilone B: Discovery of a Surprising Long-Range Effect on the Diastereoselectivity of an Aldol Condensation.” Journal of the American Chemical Society 121(30): 7050-7062.

Lee, F. Y., Borzilleri, R., et al. (2001). “BMS-247550: a novel epothilone analog with a mode of action similar to paclitaxel but possessing superior antitumor efficacy.” Clin Cancer Res 7(5): 1429-37.

Martin, N. and Thomas, E. J. (2001). “Total syntheses of epothilones B and D: applications of allylstannanes in organic synthesis.” Tetrahedron Letters 42(47): 8373-8377.

McDaid, H. M., Mani, S., et al. (2002). “Validation of the Pharmacodynamics of BMS-247550, an Analogue of Epothilone B, during a Phase I Clinical Study.” Clin Cancer Res 8(7): 2035-43.

Muhlradt, P. F. and Sasse, F. (1997). “Epothilone B stabilizes microtubuli of macrophages like taxol without showing taxol-like endotoxin activity.” Cancer Res 57(16): 3344-6.

Oza, A., Zamek, R. M., et al. (2000). “A phase I and pharmacologic trial of weekly epothilone B in patients with advanced malignancies.” Annals of Oncology 11 (Suppl.4): 133.

Service, R. F. (1996). “Tumor-killer made; how does it work?” Science 274(5295): 2009.

Su, D. -S., Meng, D., et al. (1997). “Total synthesis of (−)-epothilone B: an extension of the Suzuki coupling method and insights into structure-activity relationships of the epothilones.” Angewandte Chemie, International Edition in English 36(7): 757-759.

Winkler, J. D. and Axelsen, P. H. (1996). “A model for the taxol (paclitaxel)/epothilone pharmacophore.” Bioorganic & Medicinal Chemistry Letters 6(24): 2963-2966.

Yamaguchi, H., Paranawithana, S. R., et al. (2002). “Epothilone B analogue (BMS-247550)-mediated cytotoxicity through induction of Bax conformational change in human breast cancer cells.” Cancer Res 62(2): 466-71.

What is claimed:

1. A method to provide an antitumor treatment to a tumor-bearing subject, comprising: administering a composition containing a therapeutically effective amount of epothilone D to such subject by intravenous infusion.

2. The method of claim 1 wherein the concentration of epothilone D in said composition is between about 0.25 mg/mL and about 2.0 mg/mL.

3. The method of claim 2 wherein the concentration of epothilone D in said composition is between about 0.5 mg/mL and about 1.0 mg/mL.

4. The method of claim 3 wherein the concentration of epothilone D in said composition is about 0.5 mg/mL.

5. The method of claim 1 wherein the amount of epothilone D administered in said step of administering by intravenous infusion is at least about 100 mg of epothilone D per square meter of the surface area of such subject.

6. The method of claim 5 wherein the amount of epothilone D administered in said step of administering by intravenous infusion is at least about 120 mg of epothilone D per square meter of the surface area of such subject.

7. The method of claim 1 wherein said administering by intravenous infusion is performed for less than about 6 hours.

8. The method of claim 13, further including providing a treatment cycle comprising performing said step of administering by intravenous infusion at least once about every seven days throughout a delivery period of about twenty-one consecutive days.

9. The method of claim 8, further including repeating said step of administering by intravenous infusion twice over about fourteen days throughout said delivery period of about twenty-one consecutive days.

10. The method of claim 8, further including the step of evaluating the status of such subject to determine whether to administer additional epothilone D to such subject.

11. The method of claim 10, wherein said treatment cycle has a duration of about twenty-eight days.

12. The method of claim 11, wherein said delivery period begins on the first day of said treatment cycle.

13. The method of claim 11, further including the step of repeating said treatment cycle after the completion of said treatment period.

14. The method of claim 1, further including the step of providing to such subject a treatment cycle comprising performing said step of administering by intravenous infusion once about every twenty-four hours throughout a delivery period of about seventy-two hours.

15. The method of claim 14, wherein said treatment cycle has a duration of about fourteen consecutive days.

16. The method of claim 15, further comprising repeating said treatment cycle two times over about twenty-eight consecutive days.

17. The method of claim 16, wherein the amount of epothilone D administered in said step of administering by intravenous infusion is at least about 40 mg of epothilone D per square meter of the surface area of such subject.

18. The method of claim 17, wherein the amount of epothilone D administered in said step of administering by intravenous infusion is at least about 50 mg of epothilone D per square meter of the surface area of such subject.

19. The method of claim 16, wherein said step of administering by intravenous infusion is performed over a period of less than about two hours.

20. A method to provide an antitumor treatment to a tumor-bearing subject, comprising: administering a composition containing a therapeutically effective amount of epothilone D to such subject by intravenous infusion, wherein said intravenous infusion is performed continuously for a period of about twenty-four hours.

21. The method of claim 20, wherein said step of administering includes providing a loading dose.

22. The method of claim 21, wherein said loading dose is followed by a continuous infusion.

23. The method of claim 22, wherein said step of administering delivers a dose of less than about 250 mg of said epothilone D to such subject.

24. The method of claim 22, wherein said step of administering delivers a dose of about 70 mg of said epothilone D to such subject.

25. The method of claim 24, wherein said step of administering delivers a dose of about 200 mg of said epothilone D to such subject.
